(19) United States
(12) Patent Application Publication
(10) Pub. No.: US 2023/0284941 A1
(43) Pub. Date: Sep. 14, 2023

(54) DETECTION DEVICE

(71) Applicant: Japan Display Inc., Tokyo (JP)

(72) Inventor: Gen KOIDE, Tokyo (JP)

(21) Appl. No.: 18/113,954

(22) Filed: Feb. 24, 2023

(30) Foreign Application Priority Data
Mar. 14, 2022 (JP) 2022-039145

(51) Int. Cl. A61B 5/1455

(52) U.S. Cl. CPC A61B 5/14552; A61B 2562/0238; A61B 2562/046

(57) ABSTRACT

A detection device includes a sensor unit that has a detection area divided into a plurality of partial detection areas, a control circuit that has a first mode of acquiring data for each of the partial detection areas based on a detection value acquired from the partial detection area and a second mode of selecting the partial detection areas in which a signal strength of the data acquired in the first mode is relatively higher among the partial detection areas, and acquiring biometric data of an object to be detected based on a detection value acquired by coupling the selected partial detection areas in parallel, and an amplifying circuit configured to receive the detection value acquired in the second mode.

### CROSS-REFERENCE TO RELATED APPLICATION

This application claims the benefit of priority from Japanese Patent Application No. 2022-039145 filed on Mar. 14, 2022, the entire contents of which are incorporated herein by reference.

### BACKGROUND

### 1. Technical Field

The present disclosure relates to a detection device.

### 2. Description of the Related Art

United States Patent Application Publication No. 2018/0012069 describes an optical sensor in which a plurality of photoelectric conversion elements such as photodiodes are arranged on a semiconductor substrate. In the optical sensor, signals output from the photoelectric conversion elements change with an amount of irradiating light, thereby enabling detection of biometric information.

Japanese Patent Application Laid-open Publication No. 2019-180861 describes a configuration to acquire an oxygen saturation level in blood (hereinafter, called “blood oxygen saturation level” (SpO₂)) using pulse waves acquired using infrared light and pulse waves acquired using red light. The blood oxygen saturation level (SpO₂) refers to a ratio of an amount of oxygen actually bound to hemoglobin to the total amount of oxygen under the assumption that the oxygen is bound to all the hemoglobin in the blood.

For example, when acquiring subcutaneous information such as the pulse waves, accurate data may not always be acquired depending on the distribution of subcutaneous blood vessels. When accurately acquiring time-variable data such as the pulse waves, the detection needs to be performed at a higher frame rate. While it is conceivable to extract the highly accurate data from data acquired by a plurality of optical sensors, the configuration including the multiple optical sensors may reduce the frame rate for acquiring detection values of the multiple optical sensors. Furthermore, the computational load for calculating the pulse waves and the blood oxygen saturation level (SpO₂) may increase.

It is an object of the present disclosure to provide a detection device capable of achieving a higher frame rate of detection operation and a reduction in load of arithmetic processing in a configuration including a plurality of optical sensors.

### SUMMARY

A detection device according to an embodiment of the present disclosure includes a sensor unit that has a detection area divided into a plurality of partial detection areas, a control circuit that has a first mode of acquiring data for each of the partial detection areas based on a detection value acquired from the partial detection area and a second mode of selecting the partial detection areas in which a signal strength of the data acquired in the first mode is relatively higher among the partial detection areas, and acquiring biometric data of an object to be detected based on a detection value acquired by coupling the selected partial detection areas in parallel, and an amplifying circuit configured to receive the detection value acquired in the second mode.

### BRIEF DESCRIPTION OF THE DRAWINGS

FIG. 1 is a plan view illustrating a detection device according to an embodiment;

FIG. 2 is a view illustrating an exemplary detection area;

FIG. 3 is a conceptual diagram illustrating an exemplary basic configuration for explaining a basic detection operation of the detection device;

FIG. 4 is a timing waveform diagram illustrating a basic detection operation example of the detection device;

FIG. 5 is a block diagram illustrating an exemplary circuit configuration of the detection device according to a first example of a comparative example;

FIG. 6 is a block diagram illustrating an exemplary circuit configuration of the detection device according to a second example of the comparative example;

FIG. 7A is a timing waveform diagram illustrating an exemplary detection operation of the detection device according to the comparative example;

FIG. 7B is a timing waveform diagram illustrating another exemplary detection operation of the detection device according to the comparative example;

FIG. 8A is a diagram illustrating an exemplary pulse waveform acquired in each partial detection area;

FIG. 8B is a diagram illustrating another exemplary pulse waveform acquired in each partial detection area;

FIG. 8C is a diagram illustrating still another exemplary pulse waveform acquired in each partial detection area;

FIG. 9A is a view illustrating a first example of the detection area according to a first embodiment;

FIG. 9B is a view illustrating a second example of the detection area according to the first embodiment;

FIG. 9C is a view illustrating a third example of the detection area according to the first embodiment;

FIG. 10 is a block diagram illustrating a circuit configuration example of the detection device according to the first embodiment;

FIG. 11 is a flowchart illustrating an exemplary detection process flow in the detection device according to the first embodiment;

FIG. 12 is a timing waveform diagram illustrating an operation example in an entire detection area data acquisition process of the detection device according to the first embodiment;

FIG. 13 is a view illustrating a selection state in a partial detection area selection process of the detection device according to the first embodiment;

FIG. 14 is a timing waveform diagram illustrating an operation example in a partial detection area setting process of the detection device according to the first embodiment;

FIG. 15 is a timing waveform diagram illustrating an operation example in a pulse wave data acquisition process of the detection device according to the first embodiment;

FIG. 16 is a diagram illustrating an exemplary circuit configuration of a detection circuit according to the first embodiment;

FIG. 17 is a block diagram illustrating a circuit configuration example of the detection device according to a modification of the first embodiment;

FIG. 18 is a diagram illustrating an exemplary schematic configuration of the detection device according to a second embodiment;

FIG. 19 is a block diagram illustrating a circuit configuration example of a partial detection area according to the second embodiment;

FIG. 20 is a diagram illustrating an exemplary specific circuit configuration of a first drive circuit;

FIG. 21 is a diagram illustrating an exemplary specific circuit configuration of a second drive circuit;

FIG. 22 is a timing waveform diagram illustrating an operation example in the entire detection area data acquisition process of the detection device according to the second embodiment;

FIG. 23 is a timing waveform diagram illustrating an operation example in the partial detection area setting process of the detection device according to the second embodiment;

FIG. 24 is a conceptual diagram illustrating examples of DATA signals that are set in the partial detection area setting process of the detection device according to the second embodiment;

FIG. 25 is a timing waveform diagram illustrating an operation example in the pulse wave data acquisition process of the detection device according to the first embodiment; and

FIG. 26 is a block diagram illustrating a circuit configuration example of the partial detection area according to a modification of the second embodiment.

### DETAILED DESCRIPTION

The following describes modes (embodiments) for carrying out the present disclosure in detail with reference to the drawings. The present disclosure is not limited to the description of the embodiments to be given below. Components to be described below include those easily conceivable by those skilled in the art or those substantially identical thereto. In addition, the components to be described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the description and the drawings, and detailed description thereof may not be repeated where appropriate.

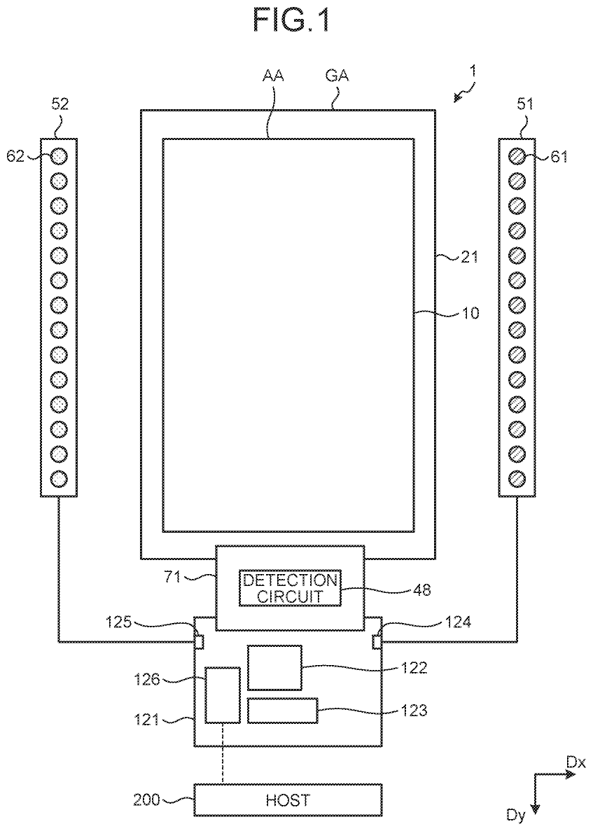

FIG. 1 is a plan view illustrating a detection device according to an embodiment. As illustrated in FIG. 1, a detection device 1 includes a sensor base material 21, a sensor unit 10, a detection circuit 48, a control circuit 122, a power supply circuit 123, first light sources 61, and second light sources 62. FIG. 1 illustrates an example in which a first light source base material 51 is provided with the first light sources 61 and a second light source base material 52 is provided with the second light sources 62. However, the arrangement of the first and the second light sources 61 and 62 illustrated in FIG. 1 is merely an example, and can be changed as appropriate. For example, the first and the second light sources 61 and 62 may be arranged on each of the first and the second light source base materials 51 and 52. In this case, a group including the first light sources 61 and a group including the second light sources 62 may be arranged in a second direction Dy, or the first and the second light sources 61 and 62 may be alternately arranged in the second direction Dy. The first and the second light sources 61 and 62 may be provided on one light source base material, or three or more light source base materials. A specific example of the arrangement of the first and the second light sources 61 and 62 will be described later.

The detection device 1 is electrically coupled to a host 200. The host 200 is, for example, a higher-level control device for an apparatus (not illustrated) to which the detection device 1 is applied. The host 200 performs a predetermined biometric information acquisition process based on data output from the detection device 1.

The sensor base material 21 is electrically coupled to a control substrate 121 through a flexible printed circuit board 71. The flexible printed circuit board 71 is provided with the detection circuit 48. The control substrate 121 is provided with the control circuit 122, the power supply circuit 123, and an output circuit 126.

The detection circuit 48 is, for example, an analog front-end (AFE) circuit. The detection circuit 48 detects output values of the sensor unit 10.

The control circuit 122 includes, for example, a control integrated circuit (IC) that outputs logic control signals. The control circuit 122 may be, for example, a programmable logic device (PLD) such as a field-programmable gate array (FPGA).

The control circuit 122 supplies control signals to the sensor unit 10 and the detection circuit 48. The control circuit 122 supplies control signals to the first and the second light sources 61 and 62. In the present disclosure, the control circuit 122 is a component that executes each process of the overall detection operation in the detection device 1, including an entire detection area data acquisition process, a partial detection area selection process, a partial detection area setting process, and a pulse wave data acquisition process, which are to be described later, based on detection values acquired by the detection circuit 48.

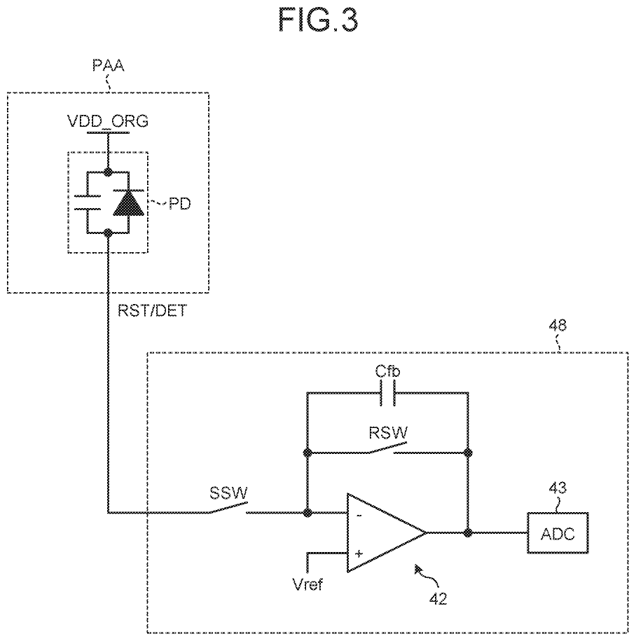

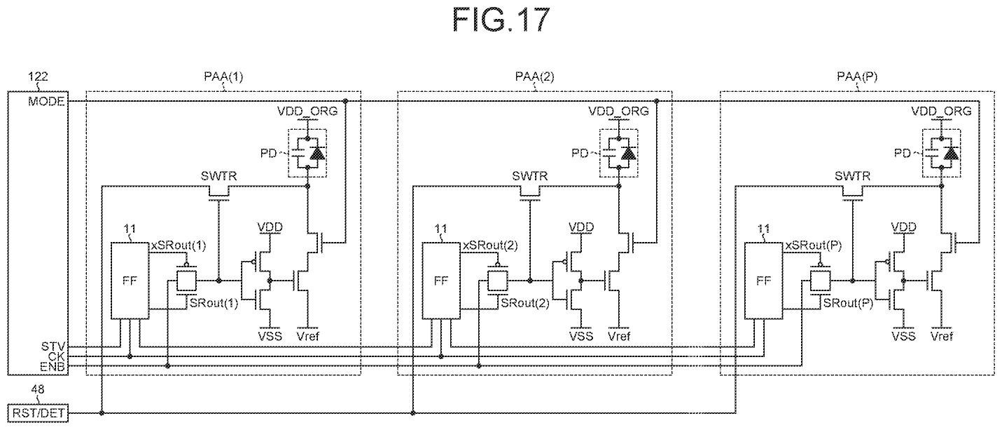

The power supply circuit 123 supplies power supply voltages for components, including, for example, a sensor power supply potential VDD_ORG, a reference potential Vref (refer to FIG. 3, for example), a control high potential VDD, and a control low potential VSS (refer to FIG. 17, for example), to the sensor unit 10 and detection circuit 48. The power supply circuit 123 supplies a light source drive voltage to the first and the second light sources 61 and 62.

The output circuit 126 is, for example, a Universal Serial Bus (USB) controller IC, and controls communication between the control circuit 122 and the host 200.

The sensor base material 21 has a detection area AA and a peripheral area GA. The detection area AA is an area provided with a plurality of optical sensors PD (refer to FIG. 3) included in the sensor unit 10. The peripheral area GA is an area between the outer perimeter of the detection area AA and the ends of the sensor base material 21, and is an area not provided with the optical sensors PD.

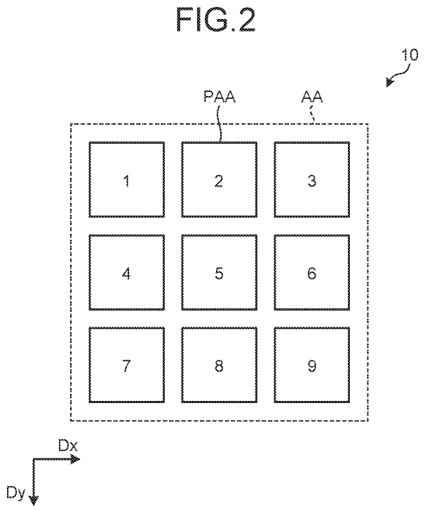

FIG. 2 is a view illustrating an example of the detection area. The detection area AA is divided into a plurality of partial detection areas PAA, and each of the partial detection areas PAA is provided with a corresponding one of the optical sensors PD. In the example illustrated in FIG. 2, the detection area AA is divided into nine of the partial detection areas PAA by being divided into three areas in the first direction Dx and three areas in the second direction Dy.

The first direction Dx is one direction in a plane parallel to the sensor base material 21. The second direction Dy is one direction in the plane parallel to the sensor base material 21, and is a direction orthogonal to the first direction Dx. The second direction Dy may non-orthogonally intersect the first direction Dx. A third direction Dz is a direction orthogonal to the first direction Dx and the second direction Dy, and is a direction normal to the sensor base material 21.

The first light sources 61 are provided on the first light source base material 51, and are arranged along the second direction Dy. The second light sources 62 are provided on the second light source base material 52, and are arranged along the second direction Dy. The first light source base material 51 and the second light source base material 52 are electrically coupled, through terminals 124 and 125, respectively, provided on the control substrate 121, to the control circuit 122 and the power supply circuit 123.

For example, inorganic light-emitting diodes (LEDs) or organic electroluminescent (EL) diodes (organic light-emitting diodes (OLEDs)) are used as the first and the second light sources 61 and 62. The first and the second light sources 61 and 62 emit first light and second light, respectively, having different wavelengths.

The first light emitted from the first light sources 61 is reflected, for example, on a surface of an object to be detected, such as a finger or a wrist of a subject, and is incident on the sensor unit 10. As a result, the sensor unit 10 can detect a fingerprint by detecting a shape of asperities on a surface of a finger Fg or the like. The second light emitted from the second light sources 62 is, for example, reflected in the finger Fg or the like, or transmitted through the finger Fg or the like, and is incident on the sensor unit 10. As a result, the sensor unit 10 can detect information on a living body in the finger, the wrist, and the like of the subject. Examples of the information on the living body include pulse waves, pulsation, and a vascular image of the subject. That is, the detection device 1 may be configured as a fingerprint detection device to detect the fingerprint or a vein detection device to detect a vascular pattern of, for example, veins.

The first light may have a wavelength of from 520 nm to 600 nm, for example, a wavelength of approximately 500 nm, and the second light may have a wavelength of from 780 nm to 950 nm, for example, a wavelength of approximately 850 nm. In this case, the first light is visible light in blue or green (blue light or green light), and the second light is infrared light. The sensor unit 10 can detect the fingerprint based on the first light emitted from the first light sources 61. The second light emitted from the second light sources 62 is reflected in, or transmitted through or absorbed by the object to be detected, and is incident on the sensor unit 10. As a result, the sensor unit 10 can detect the biometric data such as the pulse waves and the vascular image (vascular pattern) as the information on the living body in the finger, the wrist, and the like of the subject.

Alternatively, the first light may have a wavelength of from 600 nm to 700 nm, for example, approximately 660 nm, and the second light may have a wavelength of from 780 nm to 950 nm, for example, approximately 850 nm. In this case, the sensor unit 10 can detect a blood oxygen level in addition to the pulse waves, the pulsation, and the vascular image as the information on the living body based on the first light emitted from the first light sources 61 and the second light emitted from the second light sources 62. As described above, the detection device 1 includes the first and the second light sources 61 and 62, and performs the detection based on the first light and the detection based on the second light, and thereby can detect the various types of information on the living body.

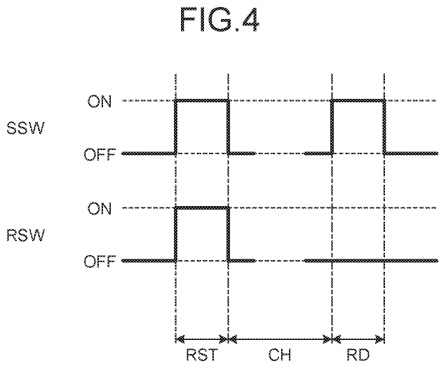

FIG. 3 is a conceptual diagram illustrating an exemplary basic configuration for explaining a basic detection operation of the detection device. FIG. 4 is a timing waveform diagram illustrating a basic detection operation example of the detection device;

Each of the optical sensors PD provided in the partial detection area PAA is an organic photodiode (OPD), and outputs an electrical signal corresponding to light emitted thereto to the detection circuit 48.

The detection circuit 48 includes an amplifying circuit 42 and an analog-to digital (A/D) conversion circuit 43 (hereinafter also called “ADC 43”) as a basic configuration for performing the detection operation.

As illustrated in FIG. 4, the detection device 1 includes a reset period RST, an exposure period CH, and a read period RD. The detection device 1 performs the detection operation through a sequence of transition processes of the reset period RST, the exposure period CH, and the read period RD to be described below.

Specifically, the power supply circuit 123 supplies the sensor power supply potential VDD_ORG to the cathode of the optical sensor PD over the reset period RST, the exposure period CH, and the read period RD. The first light (or the second light) is emitted from the first light sources 61 (or the second light sources 62) over the reset period RST, the exposure period CH, and the read period RD.

The control circuit 122 controls a switch RSW and a switch SSW of the detection circuit 48 to be on during the reset period RST. As a result, the reference potential Vref applied to the non-inverting input (+) of the amplifying circuit 42 is supplied to the anode of the optical sensor PD, and thus, a reverse bias is applied between the anode and the cathode of the optical sensor PD. At this time, the optical sensor PD is charged with an electric charge corresponding to the reverse bias voltage.

The control circuit 122 controls the switch SSW to be off during the exposure period CH. This operation gradually discharges the electric charge with which the optical sensor PD has been charged by the light irradiation.

The control circuit 122 controls the switch SSW to be on, and the switch RSW to be off during the read period RD. This operation moves the electric charge that has charged the optical sensor PD to a negative feedback capacitor Cfb of the amplifying circuit 42 in the detection circuit 48, and sets the output voltage of the amplifying circuit 42 to a voltage corresponding to the electric charge stored in the negative feedback capacitor Cfb. The output voltage of the amplifying circuit 42 is converted into a digital value by the ADC 43.

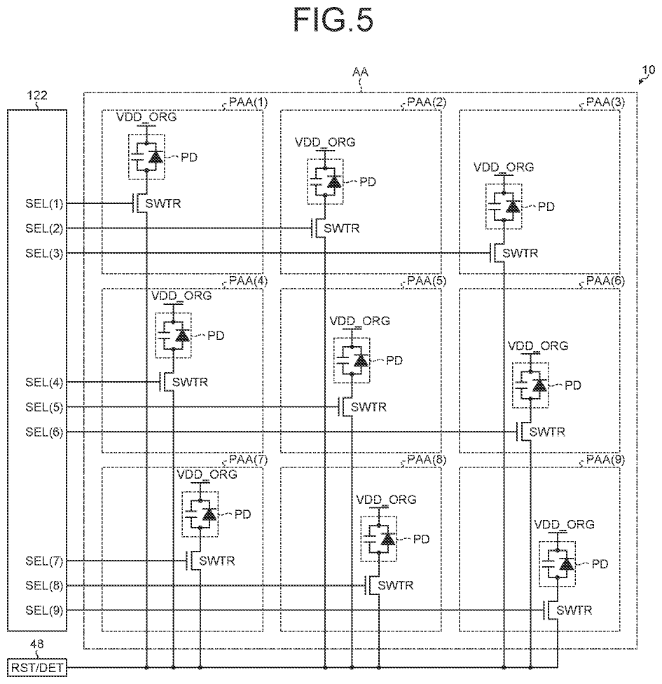

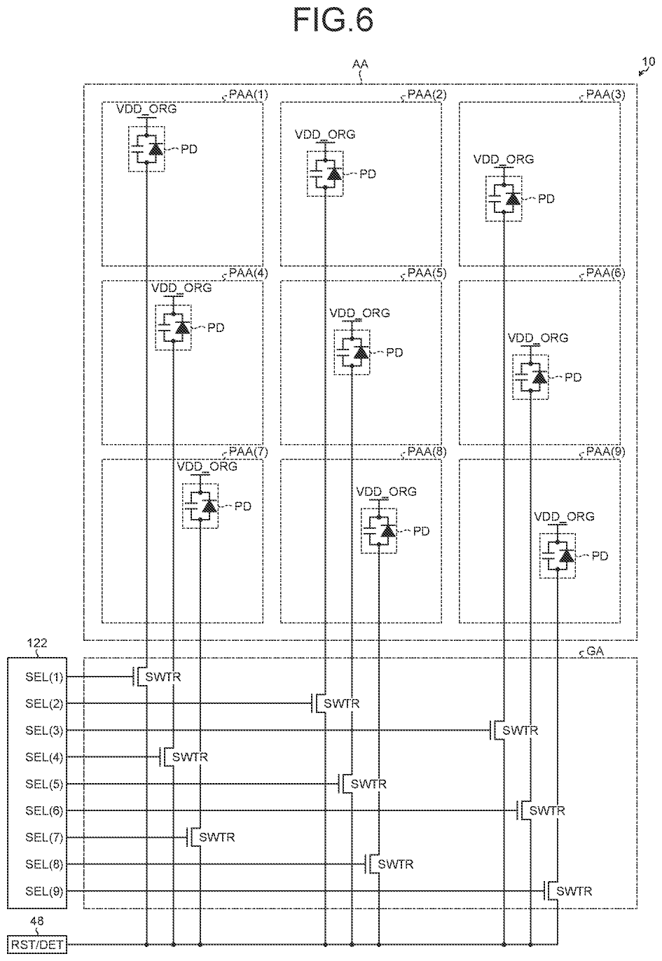

FIG. 5 is a block diagram illustrating an exemplary circuit configuration of the detection device according to a first example of a comparative example. FIG. 6 is a block diagram illustrating an exemplary circuit configuration of the detection device according to a second example of the comparative example. In FIGS. 5 and 6, the sensor unit 10 has the nine partial detection areas PAA obtained by dividing the detection area AA into three areas in the first direction Dx and three areas in the second direction Dy.

As illustrated in FIGS. 5 and 6, in the comparative example, the anodes of the optical sensors PD provided in the partial detection areas PAA(1), PAA(2), . . . , PAA(9) are coupled into a bundle, each via a switch transistor SWTR, and are coupled to the detection circuit 48. FIG. 5 illustrates the example where the switch transistor SWTR is provided in each of the partial detection areas PAA(1), PAA(2), . . . , PAA(9). However, in an aspect of the present disclosure, the switch transistors SWTR may be provided in the peripheral area GA as illustrated in FIG. 6.

In the comparative example illustrated in FIGS. 5 and 6, the gates of the switch transistors SWTR are supplied with SEL (SEL(1), SEL(2), . . . , SEL(9)) signals from the control circuit 122.

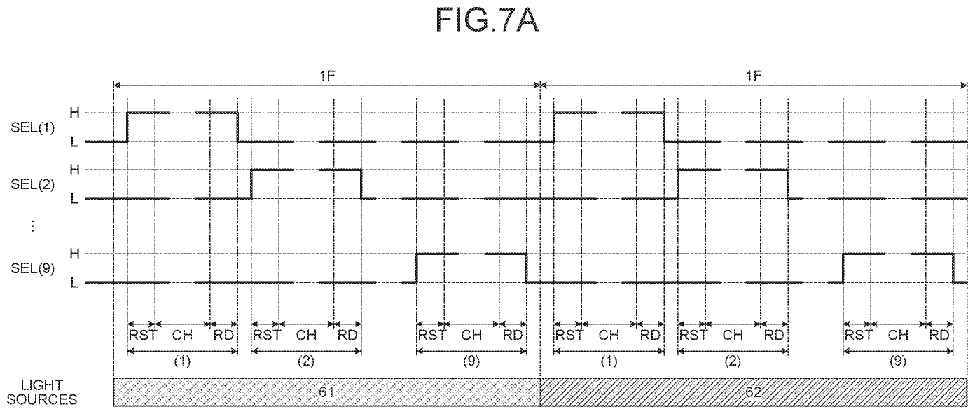

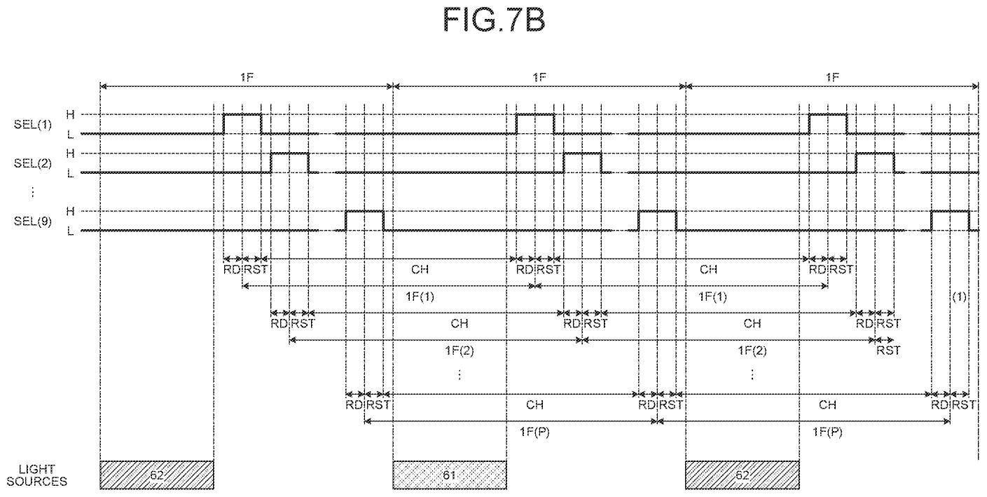

The detection operation in the comparative example illustrated in FIGS. 5 and 6 will be described with reference to FIGS. 7A and 7B. FIGS. 7A and 7B are timing waveform diagrams illustrating exemplary detection operations of the detection device according to the comparative example.

The following describes an example of acquiring the pulse waves serving as biometric information for calculating an oxygen saturation level in blood (hereinafter, called “blood oxygen saturation level” (SpO₂)) as a specific example of the information on the living body acquired by the detection device 1.

In the case of acquiring the human blood oxygen saturation level (SpO₂), a pulse wave acquired using the first light (red light) and a pulse wave acquired using the second light (infrared light) are used. Specifically, for example, visible light in red (red light) having a wavelength of from 600 nm to 700 nm, specifically, approximately 660 nm is employed as the first light emitted from the first light sources 61, and infrared light having a wavelength of from 780 nm to 950 nm, specifically, approximately 850 nm is employed as the second light emitted from the second light sources 62.

In the detection operation example illustrated in FIG. 7A, the control circuit 122 sequentially controls each of the SEL signals to a high potential (hereinafter, also referred to as “H-control”) during one frame period 1F in which the first light sources 61 are turned on to emit the first light (red light), and performs the detection operation described above during the high period (hereinafter, also referred to as “H period”) of each of the SEL signals. This operation acquires first detection values DET1(1), DET1(2), . . . , DET1(9) for the partial detection areas PAA(1), PAA(2), . . . , PAA(9), respectively.

In the subsequent one frame period 1F, the control circuit 122 turns on the second light sources 62 to emit the second light (infrared light), sequentially H-controls each of the SEL signals, and performs the detection operation described above during the H period of each of the SEL signals. This operation acquires second detection values DET2(1), DET2(2), . . . , DET2(9) for the partial detection areas PAA(1), PAA(2), . . . , PAA(9), respectively.

Thus, by alternately providing the frame to turn on the first light sources 61 to emit the first light (red light) and the frame to turn on the second light sources 62 to emit the second light (infrared light), pulse wave data generated by the first detection values DET1(1), DET1(2), . . . , DET1(9) acquired using the first light (red light) and pulse wave data generated by the second detection values DET2(1), DET2(2), . . . , DET2(9) acquired using the second light (infrared light) can be used to calculate the blood oxygen saturation level (SpO₂).

Since the calculation of the blood oxygen saturation level (SpO₂) uses the pulse wave acquired using the first light (red light) and the pulse wave acquired using the second light (infrared light), the gap in detection timing between the first detection values DET1 acquired using the first light and the second detection signals DET2 acquired using the second light is preferably smaller.

However, in the detection operation example illustrated in FIG. 7A, the first detection values DET1(1), DET1(2), . . . , DET1(9) and the second detection values DET2(1), DET2(2), . . . , DET2(9) are acquired for all the partial detection areas PAA(1), PAA(2), . . . , PAA(9), respectively, in the detection area AA. Therefore, the gap in detection timing is large between the first detection values DET1 acquired in the frame to turn on the first light sources 61 to emit the first light (red light) and the second detection values DET2 acquired in the frame to turn on the second light sources 62 to emit the second light (infrared light). As the number of divisions in the detection area AA increases, the gap in detection timing becomes more significant between the first detection values DET1 acquired using the first light and the second detection signals DET2 acquired using the second light. In other words, the frame rate in the detection operation becomes more difficult to be improved as the number of divisions in the detection area AA increases, that is, as the areal density of the optical sensors PD in the detection area AA increases.

In addition, if the first detection values DET1(1), DET1(2), . . . , DET1(9) and the second detection values DET2(1), DET2(2), . . . , DET2(9) for all the partial detection areas PAA(1), PAA(2), . . . , PAA(9), respectively, in the detection area AA are transmitted to the host 200 and the host 200 calculates the blood oxygen saturation level (SpO₂), the amount of transmitted data and the load in arithmetic processing on the side of the host 200 may increase.

In contrast to the detection operation example illustrated in FIG. 7A, in the detection operation example illustrated in FIG. 7B, the read period RD and the reset period RST are sequentially provided for each of the partial detection areas PAA(p) in the one frame period 1F, and a period until the read period RD in the next frame is used as the exposure period CH. Therefore, a period provided with the read periods RD and the reset periods RST in the other partial detection areas PAA can be used as the exposure period CH. This configuration can make the one frame period shorter than that of the detection operation example illustrated in FIG. 7A

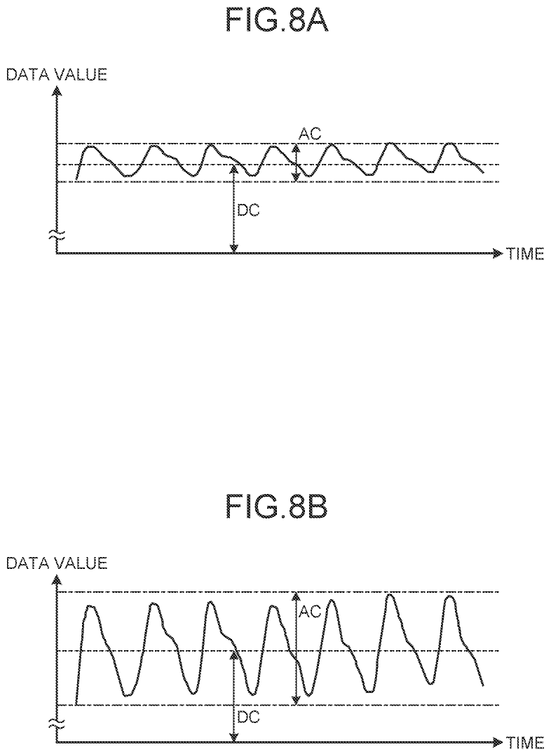

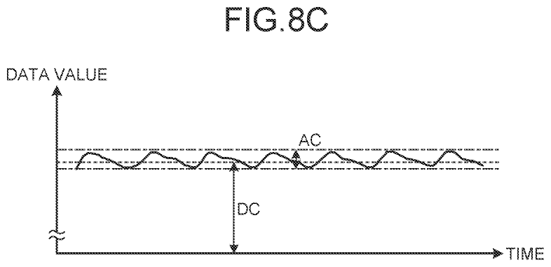

FIGS. 8A, 8B, and 8C are diagrams illustrating exemplary pulse waveforms acquired in each of the partial detection areas. In FIGS. 8A, 8B, and 8C, the horizontal axis represents time, and the vertical axis represents the data value of the pulse wave data.

The signal strength of the pulse wave data acquired in each of the partial detection areas PAA in the detection area AA differs depending on the distribution of subcutaneous blood vessels in the finger of the subject. The signal strength of the pulse wave data is represented by the alternating-current (AC) component of the pulse wave data illustrated in FIGS. 8A, 8B, and 8C. Specifically, for example, the signal strength represented by the AC component of the pulse wave data illustrated in FIG. 8B is higher relative to the signal strength represented by the AC component of the pulse wave data illustrated in FIG. 8A and the AC component of the pulse wave data illustrated in FIG. 8C.

In the present disclosure, as illustrated in, for example, FIG. 8B, the partial detection areas PAA in which the pulse wave data having a relatively higher signal strength is acquired are selected from among the partial detection areas PAA, and the optical sensors PD in the selected partial detection areas PAA are coupled in parallel and perform the detection operation. This configuration can achieve a higher frame rate of the detection operation and a reduction in the load of the arithmetic processing when acquiring the pulse wave data.

The following describes a configuration and an operation in the configuration including the optical sensors PD that can achieve a higher frame rate of the detection operation and a further reduction in the load of the arithmetic processing when acquiring the pulse wave data than the above-described detection operation of the comparative example illustrated in FIG. 7B.

### First Embodiment

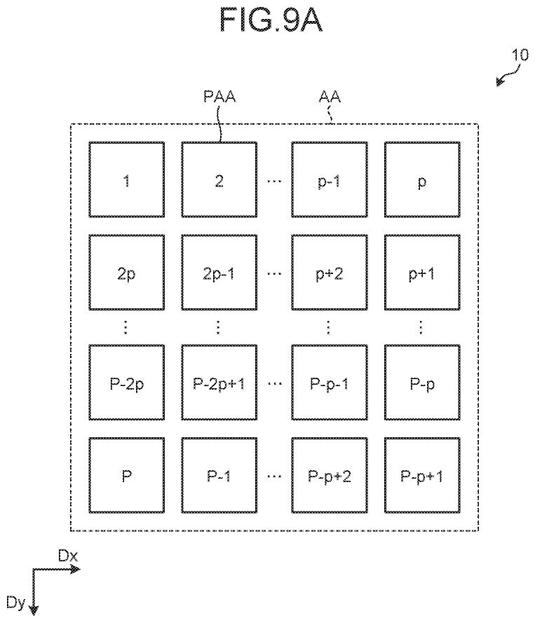

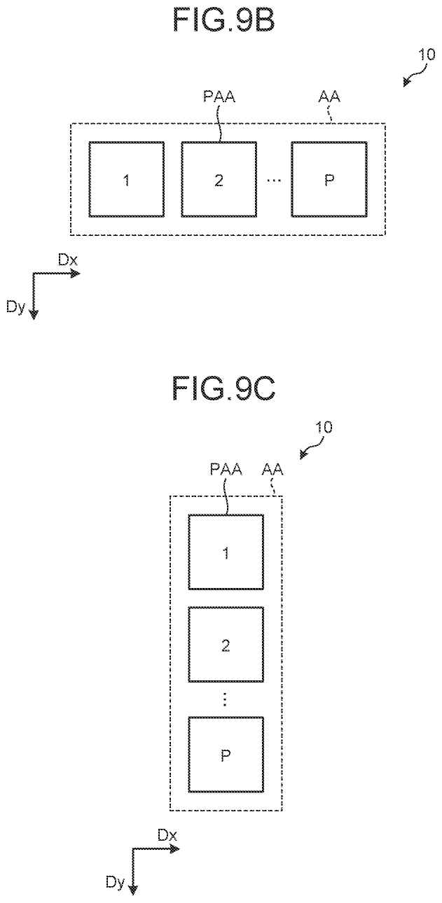

FIG. 9A is a view illustrating a first example of the detection area according to a first embodiment. FIG. 9B is a view illustrating a second example of the detection area according to the first embodiment. FIG. 9C is a view illustrating a third example of the detection area according to the first embodiment.

In the first example illustrated in FIG. 9A, the detection area AA is divided into P partial detection areas PAA by being divided into p areas in the first direction Dx and P/p areas in the second direction Dy. The mode of division of the detection area AA is not limited to that illustrated in FIG. 9A. The detection area AA may be divided in a mode of being divided into P areas in the first direction Dx as illustrated in FIG. 9B, or a mode of being divided into P areas in the second direction Dy as illustrated in FIG. 9C.

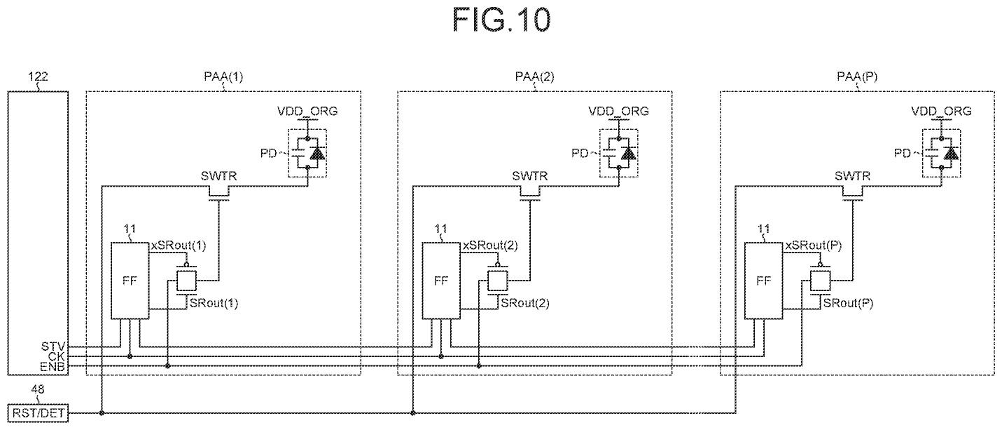

FIG. 10 is a block diagram illustrating a circuit configuration example of the detection device according to the first embodiment. As illustrated in FIG. 10, the configuration according to the first embodiment includes a flip-flop circuit 11 (hereinafter, also called “FF 11”) in each of the partial detection areas PAA. For example, a D flip-flop circuit is exemplified as the FF 11. The FFs 11 included in the partial detection areas PAA(p) are coupled in a cascade arrangement to form a shift register circuit. The configuration of the shift register circuit is not limited thereto.

The FF 11 of the partial detection area PAA(1) receives an STV signal and a CK signal. The FFs 11 of the partial detection area PAA(2) and the subsequent partial detection areas PAA receive the output of the FF 11 at the previous stage and the CK signal. The switch transistor SWTR in each of the partial detection areas PAA(p) is selectively supplied with an ENB signal by an SRout signal and an xSRout signal output from the FF 11. The STV signal, the CK signal, and the ENB signal are output from the control circuit 122. The xSRout signal is a logically inverted signal of the SRout signal.

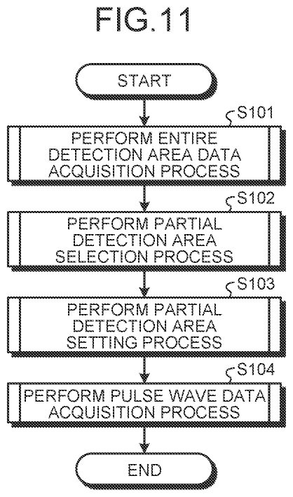

FIG. 11 is a flowchart illustrating an exemplary detection process flow in the detection device according to the first embodiment. The control circuit 122 mainly performs the entire detection area data acquisition process (Step S101), the partial detection area selection process (Step S102), the partial detection area setting process (Step S103), and the pulse wave data acquisition process (Step S104) illustrated in FIG. 11. In the present disclosure, the entire detection area data acquisition process (Step S101) corresponds to a “first mode”. In the present disclosure, the partial detection area selection process (Step S102), the partial detection area setting process (Step S103), and the pulse wave data acquisition process (Step S104) correspond to a “second mode”.

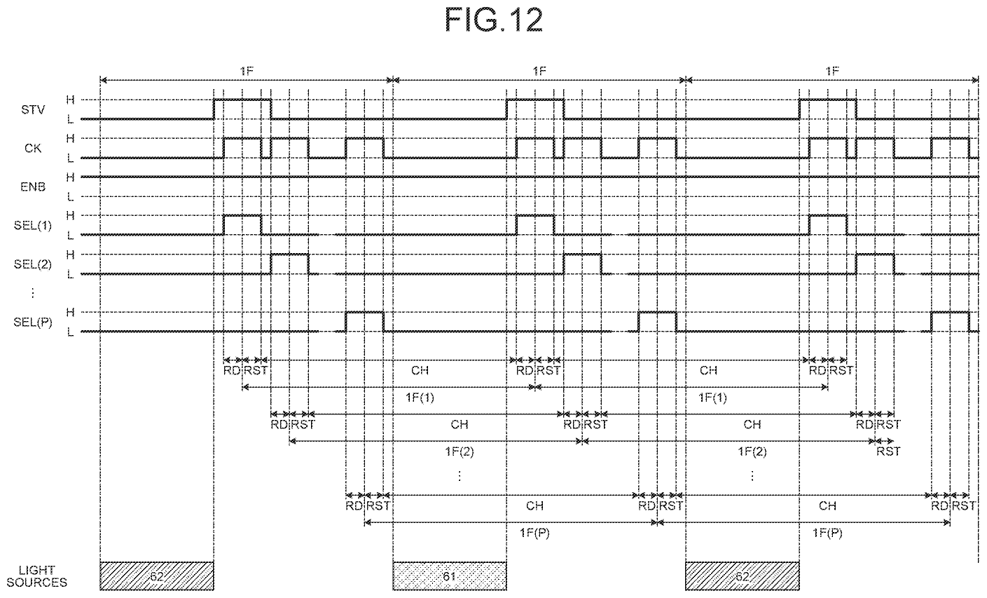

FIG. 12 is a timing waveform diagram illustrating an operation example in the entire detection area data acquisition process of the detection device according to the first embodiment. In the entire detection area data acquisition process (Step S101), the control circuit 122 continuously turns on either the first light sources 61 or the second light sources 62, and acquires the pulse wave data of all the partial detection areas PAA(p) in the detection area AA.

Specifically, the control circuit 122 acquires detection values DET(p) for a plurality of frames in each of the partial detection areas PAA(p) in the detection area AA. A number of frames F when acquiring the detection values DET(p) for a plurality of frames is set to a number of times (for example, approximately ten times) by which a peak of the pulse wave can be acquired.

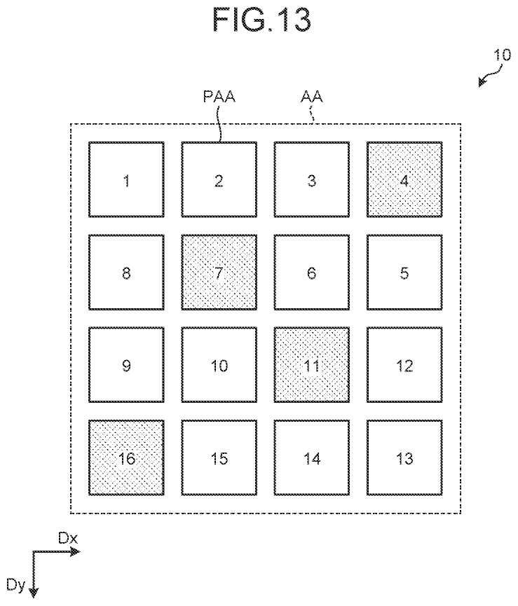

FIG. 13 is a view illustrating a selection state in the partial detection area selection process of the detection device according to the first embodiment. In the partial detection area selection process (Step S102), the control circuit 122 selects the partial detection areas PAA in which the signal strength of the acquired data is relatively higher among the partial detection areas PAA(p). FIG. 13 illustrates an example where the partial detection areas PAA(4), PAA(7), PAA(11), and PAA(16) are selected in an example where the detection area AA is divided into 16 areas.

Specifically, the control circuit 122 generates time-domain data DAT(p) that represents a time transition of the detection values DET(p) for a plurality of frames in each of the partial detection areas PAA(p) in the detection area AA. Then, based on the ratio of an AC component DAT(AC) to a direct-current (DC) component DAT(DC) of each piece of the time-domain data DAT(p) (DAT(AC)/DAT(DC)×100 [%]), the partial detection areas PAA to be used in the pulse wave data acquisition process (Step S104) are selected.

The AC component DAT(AC) of the time-domain data DAT(p) is obtained, for example, by performing a high-pass filter (HPF) process on the time-domain data DAT(p). This operation removes the DC component DAT(DC) from the time-domain data DAT(p) to obtain the AC component DAT(AC). The DC component DAT(DC) of the time-domain data DAT(p) is obtained, for example, by performing a low-pass filter (LPF) process on the time-domain data DAT(p). This operation removes the AC component DAT(AC) from the time-domain data DAT(p) to obtain the DC component DAT(DC).

The control circuit 122 selects, for example, the partial detection areas PAA in which the ratio of the AC component DAT(AC) to the DC component DAT(DC) of the time-domain data DAT(p) (DAT(AC)/DAT(DC)×100 [%]) is equal to or higher than a predetermined value (for example, 1 [%]) from among the partial detection areas PAA(p).

The mode of selection of the partial detection areas PAA in the partial detection area selection process (Step S102) is not limited thereto. The control circuit 122 may employ, for example, a mode of selecting, for example, the partial detection areas PAA in which the ratio of the AC component DAT(AC) to the DC component DAT(DC) of the time-domain data DAT(p) (DAT(AC)/DAT(DC)×100 [%]) is included in a predetermined number Q (Q=P/10, for example) of highest-ranked ratios among the partial detection areas PAA(p).

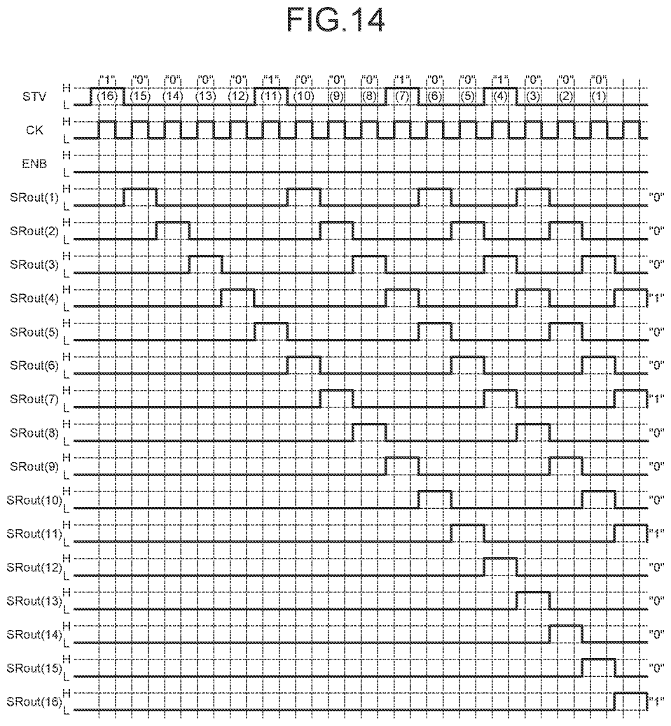

FIG. 14 is a timing waveform diagram illustrating an operation example in the partial detection area setting process of the detection device according to the first embodiment. In the partial detection area setting process (Step S103), the control circuit 122 writes a set value to the FF 11 in each of the partial detection areas PAA(p). Specifically, the control circuit 122 writes, for example, the set value “1” to the FF 11 in each of the partial detection areas (the partial detection areas PAA(4), PAA(7), PAA(11), and PAA(16) in the example illustrated in FIG. 13) selected in the partial detection area selection process (Step S102), and the set value “0” to the FF 11 of each of the other deselected partial detection areas (the partial detection areas PAA(1), PAA(2), PAA(3), PAA(5), PAA(6), PAA(8), PAA(9), PAA(10), PAA(12), PAA(13), PAA(14), and PAA(15) in the example illustrated in FIG. 13).

The control circuit 122 sets the STV signal to the set value (“0” or “1”) for each of the partial detection areas PAA(p), and sequentially outputs the STV signal. This operation writes the set value corresponding each of the FFs 11 of the partial detection areas PAA(p).

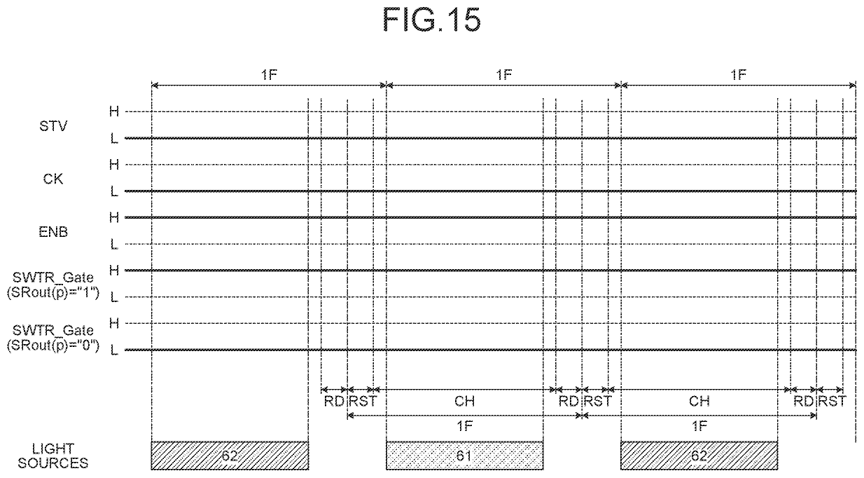

FIG. 15 is a timing waveform diagram illustrating an operation example in the pulse wave data acquisition process of the detection device according to the first embodiment. In the pulse wave data acquisition process (Step S104), the control circuit 122 controls the STV signal and the CK signal to a low potential (hereinafter, also referred to as “L-control”), and H-controls the ENB signal. This operation H-controls the switch transistor SWTR in each of the partial detection areas PAA in which “1” has been written to the FFs 11, and L-controls the switch transistor SWTR in each of the partial detection areas PAA in which “0” has been written to the FFs 11. As a result, the optical sensors PD in the partial detection areas PAA in which “1” has been written to the FFs 11 are electrically coupled in parallel, and the detection values DET obtained by superposing the detection values in the partial detection areas PAA in which “1” has been written to the FFs 11 can be acquired in the one frame period 1F.

In this state, the detection operation described above is performed by alternately providing the frame to turn on the first light sources 61 to emit the first light (red light) and the frame to turn on the second light sources 62 to emit the second light (infrared light).

Specifically, the control circuit 122 acquires the first detection values DET1 obtained by superimposing the detection values in the selected partial detection areas PAA during the one frame period 1F in which the first light sources 61 are turned on to emit the first light (red light).

The control circuit 122 also acquires the second detection values DET2 obtained by superimposing the detection values in the selected partial detection areas PAA during the one frame period 1F in which the second light sources 62 are turned on to emit the second light (infrared light).

In the pulse wave data acquisition process (Step S104), the detection values DET obtained by superimposing the detection values in the partial detection areas PAA in which “1” has been written to the FFs 11 are acquired in the one frame period 1F. This process can perform the detection operation at a higher frame rate than that of the detection operation example of the comparative example illustrated in FIG. 7B, and can reduce the amount of transmitted data and the load of the arithmetic processing when the host 200 calculates the blood oxygen saturation level (SpO₂).

In an aspect of the present disclosure, the control circuit 122 may calculate the blood oxygen saturation level (SpO₂). Even in this case, the detection operation can be performed at a higher frame rate and the load of the arithmetic processing can be lower than in the comparative example illustrated in FIG. 7B.

Furthermore, the pulse wave data acquisition process (Step S104) can improve the accuracy of acquisition of the pulse wave data and the accuracy of calculation of the blood oxygen saturation level (SpO₂) because the superimposed values of the detection values in the partial detection areas PAA are acquired.

In an aspect of the present disclosure, in the pulse wave data acquisition process (Step S104), the capacitance value of the negative feedback capacitor Cfb of the amplifying circuit 42 in the detection circuit 48 may be, for example, variable according to the number of the parallelly coupled optical sensors PD.

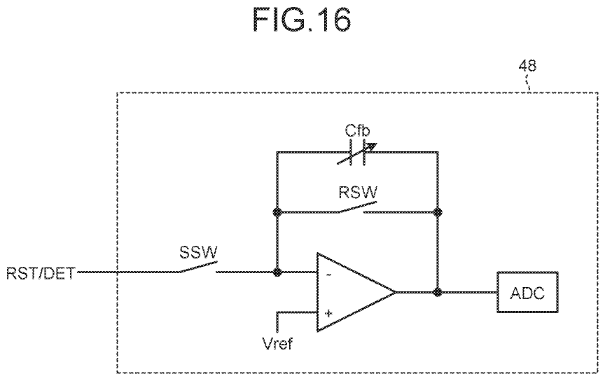

FIG. 16 is a diagram illustrating an exemplary circuit configuration of a detection circuit according to the first embodiment. In an aspect of the present disclosure, the negative feedback capacitor Cfb of the amplifying circuit 42 in the detection circuit 48 may be a variable capacitor, as illustrated in FIG. 16. The control circuit 122 controls the capacitance value of the negative feedback capacitor Cfb illustrated in FIG. 16. More specifically, in the pulse wave data acquisition process (Step S104), the capacitance value of the negative feedback capacitor Cfb is increased as the number of the parallelly coupled optical sensors PD increases.

While FIG. 16 illustrates the aspect where the negative feedback capacitor Cfb of the amplifying circuit 42 in the detection circuit 48 is a variable capacitor, for example, an aspect may be employed where a plurality of the negative feedback capacitors Cfb can be coupled in parallel, and the number of the parallelly coupled negative feedback capacitors Cfb is changed according to the number of the parallelly coupled optical sensors PD.

FIG. 17 is a block diagram illustrating a circuit configuration example of the detection device according to a modification of the first embodiment. In an aspect of the present disclosure, as illustrated in FIG. 17, the reference potential Vref may be applied to the anode of the optical sensor PD in each of the partial detection areas PAA when the partial detection area PAA is deselected. This aspect can prevent the optical sensor PD in the deselected partial detection area PAA from being brought into a floating state in the pulse wave data acquisition process (Step S104).

Specifically, when the switch transistor SWTR is L-controlled and a MODE signal is at an “H” potential, the reference potential Vref is applied to the anode of the optical sensor PD. The MODE signal is output from the control circuit 122. The MODE signal is controlled to have an “L” potential in the entire detection area data acquisition process (Step S101), and to have the “H” potential in the partial detection area setting process (Step S103) and the pulse wave data acquisition process (Step S104). This operation applies the reference potential Vref to the anode of the optical sensor PD in the deselected partial detection area PAA in the pulse wave data acquisition process (Step S104).

In an aspect of the present disclosure, in the pulse wave data acquisition process (Step S104), for example, the amount of light of the first and the second light sources 61 and 62 may be variable according to the number of the parallelly coupled optical sensors PD. The amount of light of the first and the second light sources 61 and 62 can be changed, for example, by making the light source drive voltage applied to the first and the second light sources 61 and 62 variable. Specifically, the amount of light of the first and the second light sources 61 and 62 is reduced as the number of the parallelly coupled optical sensors PD increases. The aspect is not limited to that of making the light source drive voltage applied to the first and the second light sources 61 and 62 variable, but only needs to be that where the amount of light of the first and the second light sources 61 and 62 is controllable.

In an aspect of the present disclosure, in the pulse wave data acquisition process (Step S104), for example, the exposure period CH may be variable according to the number of the parallelly coupled optical sensors PD. Specifically, the exposure period CH is shortened as the number of the parallelly coupled optical sensors PD increases. This aspect can achieve a further higher frame rate in the pulse wave data acquisition process (Step S104).

In the present disclosure, the example has been described where the pulse wave data of all the partial detection areas PAA(p) in the detection area AA is acquired in the entire detection area data acquisition process (Step S101) of the detection process flow illustrated in FIG. 11. However, in an aspect of the present disclosure, for example, the pulse wave data of all the partial detection areas PAA(p) in a predetermined area in the detection area AA may be acquired. Even in this case, the detection operation can be performed at a higher frame rate and the load of the arithmetic processing can be reduced in the pulse wave data acquisition process (Step S104) by performing the partial detection area selection process (Step S102) and the partial detection area setting process (Step S103) on all the partial detection areas PAA(p) in the predetermined area in the detection area AA.

### Second Embodiment

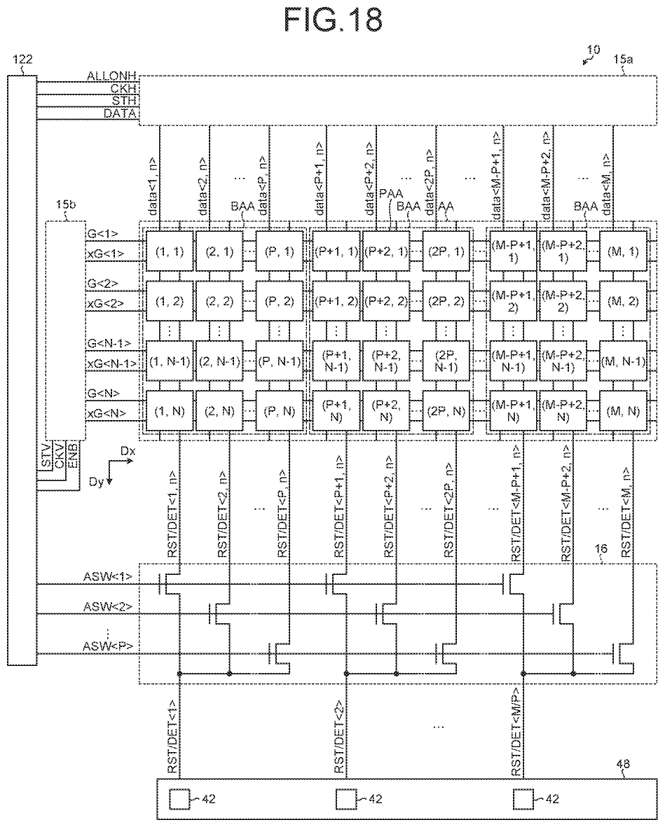

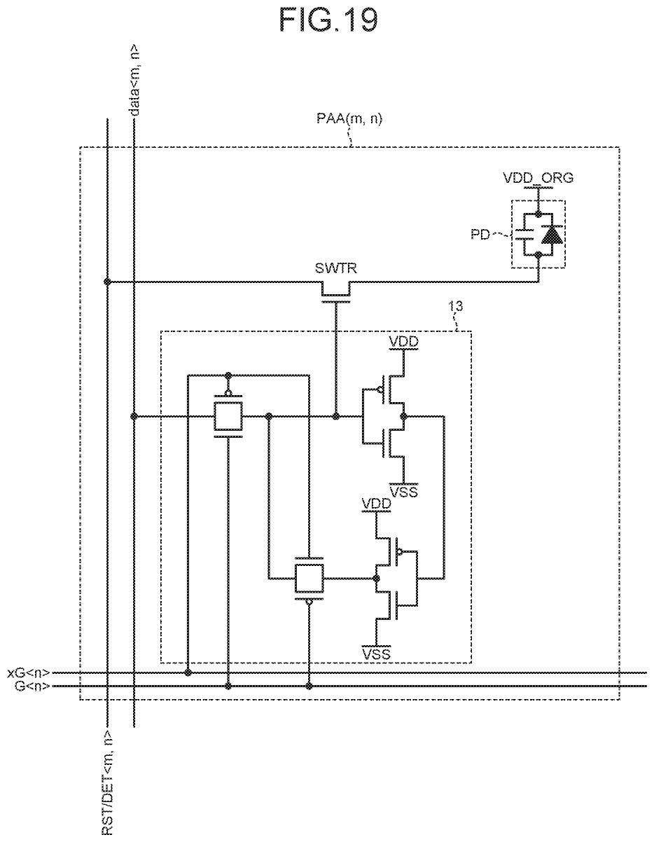

FIG. 18 is a diagram illustrating an exemplary schematic configuration of the detection device according to a second embodiment. FIG. 19 is a block diagram illustrating a circuit configuration example of a partial detection area according to the second embodiment. The detection process flow in the detection device according to the second embodiment is the same as that of the first embodiment, but the details of each process differ from those of the first embodiment.

The second embodiment has a configuration that enables acquisition of, for example, high-definition biometric data, such as the vascular image (vascular pattern), in addition to the pulse wave. As illustrated in FIG. 18, in the configuration according to the second embodiment, the detection area AA has the partial detection areas PAA arranged in a matrix having a row-column configuration. The example illustrated in FIG. 18 illustrates an example in which the detection area AA is divided into the partial detection areas PAA of M columns and N rows, where M columns of the partial detection areas PAA are arranged in the first direction Dx (row direction) and N rows of the partial detection areas PAA are arranged in the second direction Dy (column direction).

As illustrated in FIG. 18, in the configuration according to the second embodiment, the detection area AA is divided into M/P data acquisition areas BAA, in each of which P of the partial detection areas PAA are arranged in the first direction Dx (row direction).

As illustrated in FIG. 19, in the configuration according to the second embodiment, each of the partial detection areas PAA includes therein a static random access memory (SRAM) circuit 13 (hereinafter, also called “SRAM 13”). For example, a D flip-flop circuit is exemplified as the SRAM 13. In the present disclosure, the SRAM 13 corresponds to a “storage circuit”.

In the second embodiment, the sensor unit 10 includes a first drive circuit 15a, a second drive circuit 15b, and an output switching circuit 16. The first drive circuit 15a, the second drive circuit 15b, and the output switching circuit 16 are provided, for example, in the peripheral area GA.

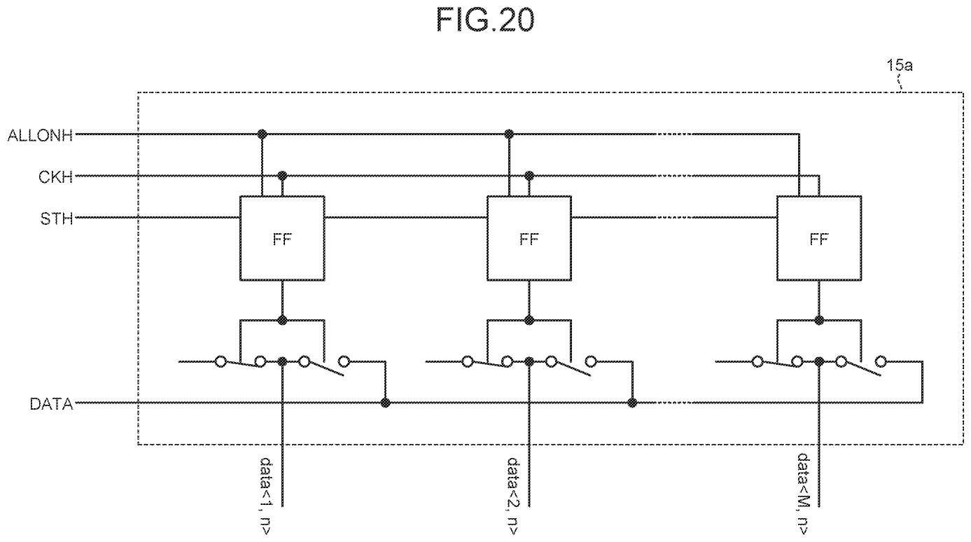

FIG. 20 is a diagram illustrating an exemplary specific circuit configuration of the first drive circuit. In the first drive circuit 15a, M flip-flop circuits corresponding to the partial detection areas PAA arranged in the second direction Dy in a column m are coupled in a cascade arrangement in the first direction Dx to form a shift register circuit. The first drive circuit 15a receives an STH signal, a CKH signal, a DATA signal, and an ALLONH signal. The STH signal, the CKH signal, the DATA signal, and the ALLONH signal are output from the control circuit 122. The ALLONH signal is controlled to have the “H” potential in the entire detection area data acquisition process (Step S101), and to have the “L” potential in the partial detection area setting process (Step S103) and the pulse wave data acquisition process (Step S104).

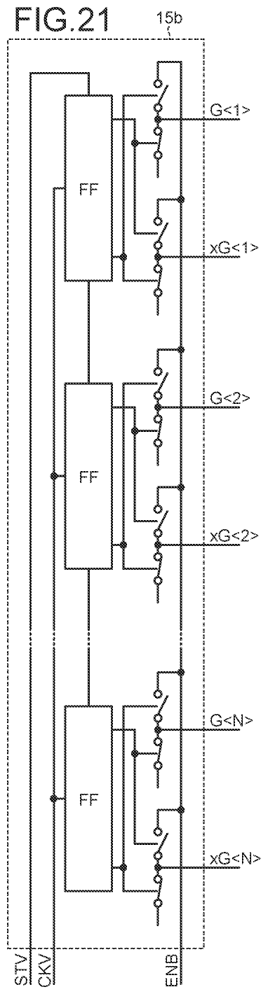

FIG. 21 is a diagram illustrating an exemplary specific circuit configuration of the second drive circuit. In the second drive circuit 15b, N flip-flop circuits corresponding to the partial detection areas PAA arranged in the first direction Dx in a row n are coupled in a cascade arrangement in the second direction Dy to form a shift register circuit. The second drive circuit 15b receives the STV signal, a CKV signal, and the ENB signal. The STV signal, the CKV signal, and the ENB signal are output from the control circuit 122. The second drive circuit 15b sequentially selects partial detection areas(m, n) in the row n based on the STV signal, the CKV signal, and the ENB signal.

Data<m, n> is selectively read by a G<n> signal and an xG<n> signal into the SRAM 13 in each of the partial detection areas PAA(m, n). The data<m, n> is output from the first drive circuit 15a. The G<n> signal and xG<n> signal are output from the second drive circuit 15b. The xG<n> signal is a logically inverted signal of the G<n> signal. When the G<n> signal is at the “H” potential and the xG<n> signal is at the “L” potential, the partial detection areas(m, n) in the row n are selected.

Specifically, in the entire detection area data acquisition process (Step S101) illustrated in FIG. 11, when the G<n> signal is H-controlled and the data<m, n> is at “H”, the switch transistor SWTR in the partial detection area PAA(m, n) is controlled to be on. In the partial detection area setting process (Step S103) illustrated in FIG. 11, when the G<n> signal is H-controlled and the data<m, n> is at “H”, the set value “1” is written to the SRAM 13 in the partial detection area PAA(m, n).

The output switching circuit 16 is provided with M switch circuits corresponding to the partial detection areas PAA arranged in the second direction Dy in the column m (refer to FIG. 18). One end of each of the M switch circuits is coupled to the partial detection areas PAA in the column m, and the other ends of the P switch circuits are bundled together and coupled to an input of each of M/P of the amplifying circuits 42. The M/P amplifying circuits 42 are provided correspondingly to the M/P data acquisition areas BAA. The output switching circuit 16 receives switching signals ASW<1>, ASW<2>, . . . , ASW<P> (where P is a natural number of M/2 or smaller). The switching signals ASW<1>, ASW<2>, . . . , ASW<P> are output from the control circuit 122.

One end of each of the switch circuits of the output switching circuit 16 is coupled to the partial detection areas PAA in the column m. The other ends of the P switch circuits of the output switching circuit 16 are bundled together and coupled to each of the M/P amplifying circuits 42. Specifically, when the number of divisions in the first direction Dx of the detection area AA is 100 and the other ends of five of the switch circuits are bundled, 20 of the amplifying circuits 42 are provided.

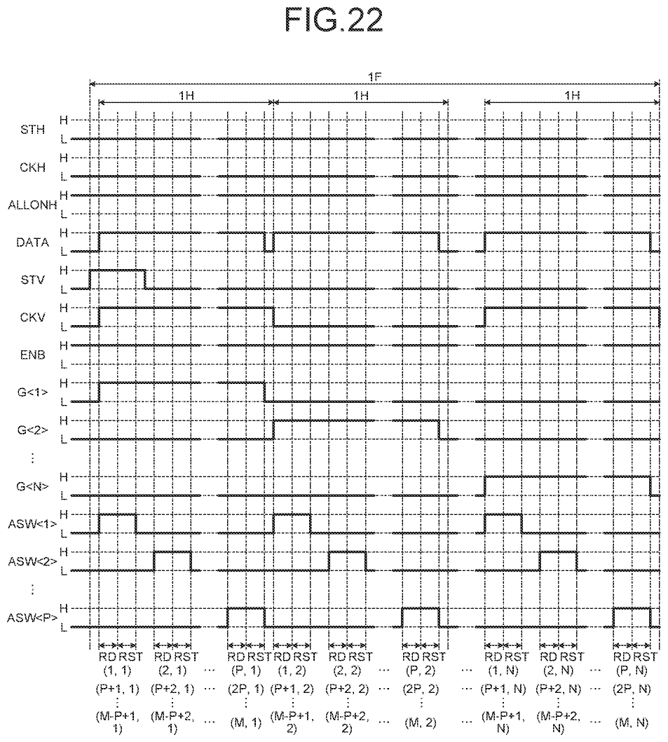

FIG. 22 is a timing waveform diagram illustrating an operation example in the entire detection area data acquisition process of the detection device according to the second embodiment. In the entire detection area data acquisition process (Step S101) illustrated in FIG. 11, the control circuit 122 continuously turns on either the first light sources 61 or the second light sources 62, and acquires the pulse wave data in all the partial detection areas PAA(m, n) in the detection area AA.

Specifically, the control circuit 122 acquires the detection values DET<m, n> for a plurality of frames in each of the partial detection areas PAA(m, n) in the detection area AA. The number of frames F when acquiring the detection values DET<m, n> for a plurality of frames is set to the number of times (for example, approximately ten times) by which a peak of the pulse wave can be acquired.

In the entire detection area data acquisition process (Step S101) illustrated in FIG. 11, the control circuit 122 H-controls the ALLONH signal, and H-controls the DATA signal for each 1H period obtained by temporally dividing the one frame period 1F into P periods. This operation sets the data<m, n> in each of the partial detection areas PAA(m, n) to the “H” potential.

In the example illustrated in FIG. 22, the G<n> signal is set to the “H” potential in each 1H period obtained by temporally dividing the one frame period 1F into the P periods. During this 1H period, the control circuit 122 sequentially H-controls the switching signals ASW<1>, ASW<2>, . . . , ASW<P>. The output switching circuit 16 simultaneously selects the partial detection areas PAA(p, n), PAA(P+p, n), . . . , PAA(M−P+p, n) based on the switching signals ASW<1>, ASW<2>, . . . , ASW<P>. The read period RD and the reset period RST are provided in the H period of each of the switching signals ASW<1>, ASW<2>, . . . , ASW<P>. Even in the present embodiment, in the same manner as in the first embodiment, a period after the period RST before the read period RD in the next frame serves as the exposure period CH in each of the partial detection areas PAA(1, n), PAA(2, n), . . . , PAA(P, n).

In the partial detection area selection process (Step S102) illustrated in FIG. 11, the control circuit 122 selects the partial detection areas PAA in which the signal strength of the data acquired by each of the M/P amplifying circuits 42 is relatively higher.

Specifically, the control circuit 122 generates the time-domain data DAT<m, n> using the detection values DET<m, n> for a plurality of frames in each of the partial detection areas PAA(m, n) in the detection area AA. Then, based on the ratio of the AC component DAT(AC) to the direct-current (DC) component DAT(DC) of each piece of the time-domain data DAT<m, n> (DAT(AC)/DAT(DC)×100 [%]), the partial detection areas PAA to be used in the pulse wave data acquisition process (Step S104) illustrated in FIG. 11 are selected. The mode of selection of the partial detection areas PAA in the partial detection area selection process (Step S102) is the same as that in the first embodiment, and therefore, will not be described in detail.

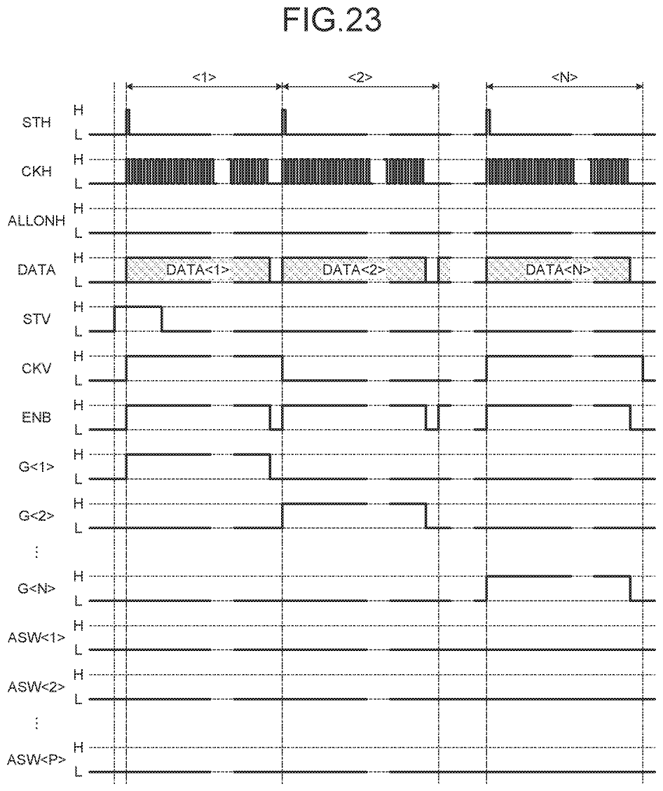

FIG. 23 is a timing waveform diagram illustrating an operation example in the partial detection area setting process of the detection device according to the second embodiment. In the partial detection area setting process (Step S103) illustrated in FIG. 11, the control circuit 122 writes the set value to the SRAM 13 in each of the partial detection areas PAA(m, n). Specifically, the control circuit 122 writes the set value “1” to the SRAM 13 in each of the partial detection areas PAA selected in the partial detection area selection process (Step S102), and writes the set value “0” to the SRAM 13 in each of the other deselected partial detection areas PAA.

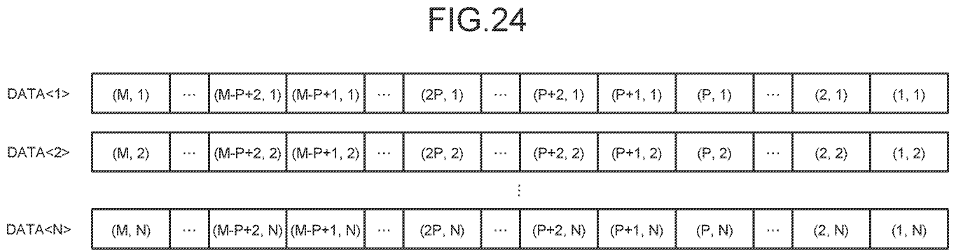

The control circuit 122 sets the DATA signal to the set value (“0” or “1”) for each of the partial detection areas PAA(m, n), and sequentially outputs the DATA signal. FIG. 24 is a conceptual diagram illustrating examples of the DATA signals that are set in the partial detection area setting process of the detection device according to the second embodiment. Each of the DATA<n> signals illustrated in FIG. 24 contains the set values (“0” or “1”) for the respective partial detection areas PAA(m, n). The DATA<n> signal corresponds to the partial detection areas PAA(m, n) in the row n sequentially selected by the second drive circuit 15b.

In the second embodiment, as illustrated in FIG. 24, the DATA<n> signal set to the set values for each row of the partial detection areas PAA(m, n) in a time-division manner is output. Specifically, for example, in an output period<1> of the DATA<1> signal, the G<1> signal is H-controlled, and as a result, the data<m, 1> corresponding to the partial detection area PAA(m, 1) is written to the SRAM 13 in the partial detection area PAA(m, 1). Also, for example, in an output period<2> of the DATA<2> signal, the G<2> signal is H-controlled, and as a result, the data<m, 2> corresponding to the partial detection area PAA(m, 2) is written to the SRAM 13 in the partial detection area PAA(m, 2). Subsequently, by repeating the same process until an output period<N> of the DATA<N> signal, the set values corresponding to the respective SRAMs 13 in the partial detection areas PAA(m, n) are written.

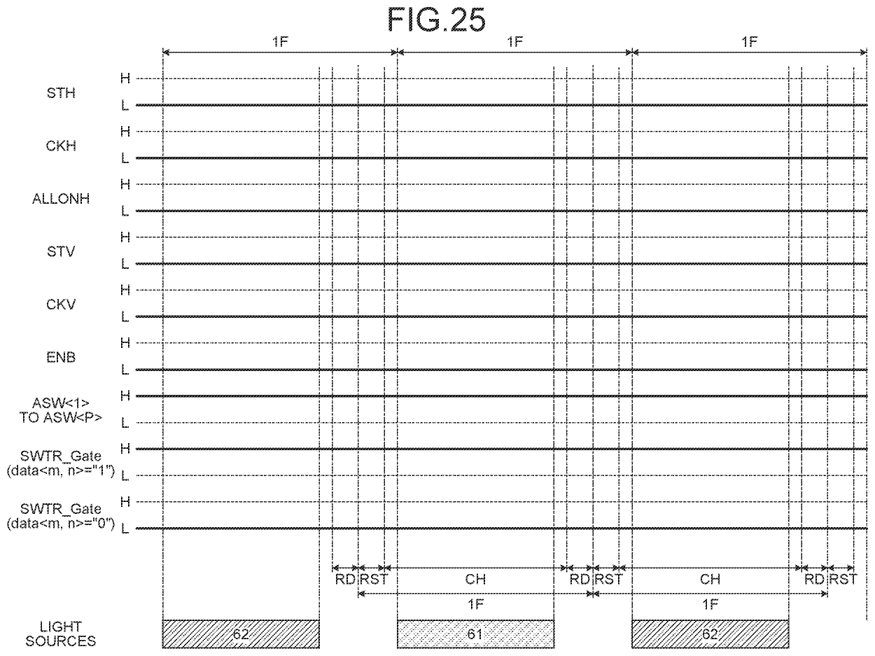

FIG. 25 is a timing waveform diagram illustrating an operation example in the pulse wave data acquisition process of the detection device according to the second embodiment. In the pulse wave data acquisition process (Step S104), the control circuit 122 L-controls the STH signal, the CKH signal, the ALLONH signal, the DATA signal, the STV signal, the CKV signal, and the ENB signal, and H-controls the ASW<p>. This operation H-controls the switch transistor SWTR in each of the partial detection areas PAA in which “1” has been written to the SRAMs 13, and L-controls the switch transistor SWTR in each of the partial detection areas PAA in which “0” has been written to the SRAMs 13. As a result, the optical sensors PD in the partial detection areas PAA in which “1” has been written to the SRAMs 13 are electrically coupled in parallel for each unit of the M/P amplifying circuits 42, and the detection values DET<1>, DET<2>, . . . , DET<M/P> for each unit of the M/P amplifying circuits 42 can be acquired.

In this state, the detection operation described above is performed by alternately providing the frame to turn on the first light sources 61 to emit the first light (red light) and the frame to turn on the second light sources 62 to emit the second light (infrared light).

Specifically, the control circuit 122 acquires the first detection values DET1 obtained by superimposing the detection values in the selected partial detection areas PAA for each unit of the M/P amplifying circuits 42 during the one frame period 1F in which the first light sources 61 are turned on to emit the first light (red light).

The control circuit 122 also acquires the second detection values DET2 obtained by superimposing the detection values in the selected partial detection areas PAA for each unit of the M/P amplifying circuits 42 during the one frame period 1F in which the second light sources 62 are turned on to emit the second light (infrared light).

In the present embodiment, in the pulse wave data acquisition process (Step S104), the detection value DET obtained by superimposing the detection values in the selected partial detection areas PAA are acquired for each unit of the M/P amplifying circuits 42 during the one frame period 1F. As a result, in the configuration that enables the acquisition of, for example, the high-definition biometric data, such as the vascular image (vascular pattern), the detection operation can be performed at a higher frame rate when acquiring the pulse wave, and the amount of transmitted data and the load of the arithmetic processing can be reduced when the host 200 calculates the blood oxygen saturation level (SpO₂). The aspect may be employed where the control circuit 122 calculates the blood oxygen saturation level (SpO₂). Even in this case, the detection operation can be performed at a higher frame rate when acquiring the pulse wave, and the load of the arithmetic processing can be reduced.

Furthermore, the pulse wave data acquisition process (Step S104) can improve the accuracy of acquisition of the pulse wave data and the accuracy of calculation of the blood oxygen saturation level (SpO₂) because the superimposed values of the detection values in the partial detection areas PAA are acquired.

Even in the present embodiment, in the same manner as in the first embodiment, the aspect may be employed where the capacitance value of the negative feedback capacitor Cfb of the amplifying circuit 42 in the detection circuit 48 is, for example, variable according to the number of the parallelly coupled optical sensors PD in the pulse wave data acquisition process (Step S104) illustrated in FIG. 11.

Specifically, for example, the aspect may be employed where the negative feedback capacitor Cfb of the amplifying circuit 42 in the detection circuit 48 is a variable capacitor, as illustrated in FIG. 16. The control circuit 122 controls the capacitance value of the negative feedback capacitor Cfb illustrated in FIG. 16. More specifically, in the pulse wave data acquisition process (Step S104) illustrated in FIG. 11, the capacitance value of the negative feedback capacitor Cfb is increased as the number of the parallelly coupled optical sensors PD increases.

For example, the aspect may also be employed where the negative feedback capacitors Cfb can be coupled in parallel, and the number of the parallelly coupled negative feedback capacitors Cfb is changed according to the number of the parallelly coupled optical sensors PD.

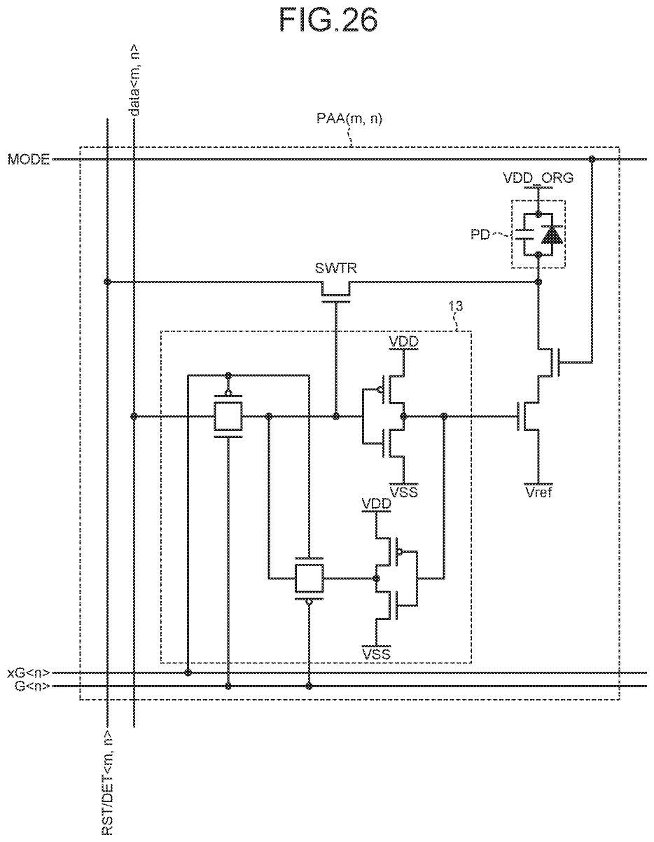

FIG. 26 is a block diagram illustrating a circuit configuration example of the partial detection area according to a modification of the second embodiment. Even in the present embodiment, in the same manner as in the first embodiment, the aspect may be employed where the reference potential Vref is applied to the anode of the optical sensor PD in each of the partial detection areas PAA when the partial detection area PAA is deselected, as illustrated in FIG. 26. This aspect can prevent the optical sensor PD in the deselected partial detection area PAA from being brought into the floating state in the pulse wave data acquisition process (Step S104) illustrated in FIG. 11.

Specifically, when the switch transistor SWTR is L-controlled and the MODE signal is at the “H” potential, the reference potential Vref is applied to the anode of the optical sensor PD. The MODE signal is output from the control circuit 122. The MODE signal is controlled to have the “L” potential in the entire detection area data acquisition process (Step S101) illustrated in FIG. 11, and to have the “H” potential in the partial detection area setting process (Step S103) and the pulse wave data acquisition process (Step S104). This operation applies the reference potential Vref to the anode of the optical sensor PD in the deselected partial detection area PAA in the pulse wave data acquisition process (Step S104).

Even in the present embodiment, in the same manner as in the first embodiment, the aspect may be employed where, for example, the amount of light of the first and the second light sources 61 and 62 is variable according to the number of the parallelly coupled optical sensors PD in the pulse wave data acquisition process (Step S104) illustrated in FIG. 11. The amount of light of the first and the second light sources 61 and 62 can be changed, for example, by making the light source drive voltage applied to the first and the second light sources 61 and 62 variable. Specifically, the amount of light of the first and the second light sources 61 and 62 is reduced as the number of the parallelly coupled optical sensors PD increases. The aspect is not limited to that of making the light source drive voltage applied to the first and the second light sources 61 and 62 variable, but only needs to be that where the amount of light of the first and the second light sources 61 and 62 is controllable.

Even in the present embodiment, in the same manner as in the first embodiment, the aspect may be employed where, for example, the exposure period CH is variable according to the number of the parallelly coupled optical sensors PD in the pulse wave data acquisition process (Step S104) illustrated in FIG. 11. Specifically, the exposure period CH is shortened as the number of the parallelly coupled optical sensors PD increases. This aspect can achieve a further higher frame rate in the pulse wave data acquisition process (Step S104).

While the preferred embodiments of the present disclosure have been described above, the present disclosure is not limited to the embodiments described above. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications described above.

What is claimed is:

1. A detection device comprising:
a sensor unit that has a detection area divided into a plurality of partial detection areas;
a control circuit that has a first mode of acquiring data for each of the partial detection areas based on a detection value acquired from the partial detection area and a second mode of selecting the partial detection areas in which a signal strength of the data acquired in the first mode is relatively higher among the partial detection areas, and acquiring biometric data of an object to be detected based on a detection value acquired by coupling the selected partial detection areas in parallel; and
an amplifying circuit configured to receive the detection value acquired in the second mode.

2. The detection device according to claim 1, wherein the control circuit is configured to select the partial detection areas in which a ratio of an alternating-current component to a direct-current component of the data acquired in the first mode is equal to or higher than a predetermined value in the second mode.

3. The detection device according to claim 1, wherein the control circuit is configured to select the partial detection areas in which a ratio of an alternating-current component to a direct-current component of the data acquired in the first mode is included in a predetermined number of highest-ranked ratios among the partial detection areas in the second mode.

4. The detection device according to claim 1, wherein the control circuit is configured to sequentially acquire the detection value for each of the partial detection areas in the first mode.

5. The detection device according to claim 4, wherein
the sensor unit comprises a shift register circuit in which flip-flop circuits provided in the respective partial detection areas are coupled in a cascade arrangement, and
the control circuit is configured to write a set value indicating a selection state in the second mode to each of the flip-flop circuits.

6. The detection device according to claim 1, wherein
the partial detection areas are provided in a matrix having a row-column configuration in the detection area,
the sensor unit comprises a storage circuit provided in each of the partial detection areas, and
the control circuit is configured to write a set value indicating a selection state in the second mode to the storage circuit.

7. The detection device according to claim 6, wherein
the sensor unit comprises:
a first drive circuit comprising a shift register circuit in which a plurality of flip-flop circuits arranged in a row direction are coupled in a cascade arrangement; and
a second drive circuit comprising a shift register circuit in which a plurality of flip-flop circuits arranged in a column direction are coupled in a cascade arrangement,
the flip-flop circuits of the first drive circuit are coupled to the respective storage circuits provided in the partial detection areas arranged in the column direction,
the flip-flop circuits of the second drive circuit are coupled to the respective storage circuits provided in the partial detection areas arranged in the row direction, and
the control circuit is configured to write the set value indicating the selection state in the second mode to the storage circuit in the partial detection area selected by the second drive circuit via the first drive circuit.

8. The detection device according to claim 6, wherein
the detection area is divided into a plurality of data acquisition areas, in each of which the partial detection areas are arranged in a row direction, and
a plurality of the amplifying circuits are provided correspondingly to the data acquisition areas.

9. The detection device according to claim 1, further comprising a light source configured to emit light to the detection area, wherein
the sensor unit comprises a plurality of optical sensors provided in the respective partial detection areas, and
the control circuit has a reset period to apply a reference potential to the optical sensors, an exposure period to expose the optical sensors to the light, and a read period to read electric charges that have charged the optical sensors.

10. The detection device according to claim 9, wherein the control circuit is configured to apply the reference potential to the optical sensors in the partial detection areas that are not selected in the second mode.

11. The detection device according to claim 9, wherein the control circuit is capable of changing a negative feedback capacitor of the amplifying circuit according to a number of the parallelly coupled partial detection areas in the second mode.

12. The detection device according to claim 9, capable of changing an amount of light of the light source according to a number of the parallelly coupled partial detection areas in the second mode.

13. The detection device according to claim 9, wherein the control circuit is capable of changing the exposure period according to a number of the parallelly coupled partial detection areas in the second mode.

14. The detection device according to claim 9, wherein
the light source comprises:
a first light source configured to emit first light to the detection area; and
a second light source configured to emit second light having a different wavelength from that of the first light to the detection area, and
the detection value acquired in the second mode comprises:
a first detection value acquired by the emission of the first light; and
a second detection value acquired by the emission of the second light.

15. The detection device according to claim 14, wherein one of the first light and the second light is infrared light.

16. The detection device according to claim 15, wherein one of the first light and the second light is red light.

17. The detection device according to claim 16, wherein the control circuit is configured to calculate a blood oxygen saturation level based on the first detection value and the second detection value that have been acquired in the second mode.

18. The detection device according to claim 9, wherein the optical sensors are organic photodiodes.
